(19) United States
(12) Patent Application Publication
(10) Pub. No.: US 2017/0349928 A1
(43) Pub. Date: Dec. 7, 2017

(54) METHODS FOR ACTIVATING NATURAL ENERGY METABOLISM FOR IMPROVING YEAST CELL-FREE PROTEIN SYNTHESIS

(71) Applicant: Northwestern University, Evanston, IL (US)

(72) Inventors: Michael Christopher Jewett, Evanston, IL (US); Mark J. Anderson, Wilmette, IL (US); Jessica Carol Stark, Evanston, IL (US); Charles Eric Hodgman, Natick, MA (US)

(73) Assignee: Northwestern University, Evanston, IL (US)

(21) Appl. No.: 15/639,364

(22) Filed: Jun. 30, 2017

Related U.S. Application Data
(63) Continuation-in-part of application No. PCT/IB2015/059960, filed on Dec. 23, 2015.
(60) Provisional application No. 62/098,578, filed on Dec. 31, 2014.

(51) Int. Cl. C12P 21/00

(52) U.S. Cl. CPC C12P 21/00

(57) ABSTRACT

Disclosed are compositions, methods, and kits for enhanced synthesis of a biological macromolecule in vitro using cell-free protein synthesis. The compositions, methods, and kits include or utilize: a cell-free extract; a phosphate-free energy source; and a phosphate source, and typically do not include an exogenous nucleoside triphosphate or an exogenous nucleoside diphosphate.

### CROSS-REFERENCE TO RELATED PATENT APPLICATIONS

The present application is a continuation-in-part of International Application No. PCT/IB2015/059960, filed on Dec. 23, 2015, published as WO 2016/108158, on Jul. 7, 2016, which claims the benefit of priority under 35 U.S.C. §119(e) to U.S. Provisional Patent Application No. 62/098,578, filed on Dec. 31, 2014, the contents of which are incorporated herein by reference in their entireties.

### STATEMENT REGARDING FEDERALLY SPONSORED RESEARCH OR DEVELOPMENT

This invention was made with government support under N66001-13-C-4024 (Leidos, Inc. subcontract to Northwestern University, No. P010152319) awarded by Space and Naval Warfare Systems Center (DARPA). The government has certain rights in the invention.

### BACKGROUND

The present invention generally relates to methods for cell-free protein synthesis. More specifically, the present invention relates to methods of activating natural energy metabolism for improving yeast cell-free protein synthesis.

Cell-free protein synthesis (CFPS) is an emerging field that allows for the production of proteins without intact cells (Carlson et al., 2012; Hodgman and Jewett, 2012). Crude cell lysates, or extracts, are employed instead. Supplying chemical energy (in the form of ATP) for the aminoacylation of tRNAs and peptide bond formation has been a grand challenge for CFPS development (Carlson et al., 2012). Historically, high-energy phosphate bond donors; such as phosphoenolpyruvate (PEP), creatine phosphate (CrP), and acetyl phosphate have been used (Brödel et al., 2013; Carlson et al., 2012; Hodgman and Jewett, 2013; Kim and Swartz, 2001; Ryabova et al., 1995; Takai et al., 2010). In these cases, ATP regeneration requires the addition of pyruvate kinase, creatine kinase, or acetate kinase, respectively, or the endogenous presence of these enzymes in the cell extract. Unfortunately, rapid production of phosphate from these high-energy compounds has been shown to be inhibitory to CFPS (e.g., E. coli (Kim and Swartz, 2000) and yeast (Schoborg et al., 2014)). Furthermore, batch reactions using these secondary energy substrates typically provide only a brief burst of ATP. In addition, phosphorylated energy compounds are costly, which limits industrial applications (Calhoun and Swartz, 2005a,g; Swartz, 2006). To address these limitations, new cost-effective secondary energy regeneration systems are sought.

Within the last decade, the E. coli CFPS platform has been able to activate natural metabolism within the lysate to fuel highly active CFPS from non-phosphorylated energy substrates and avoid costly substrates by replacing PEP with glucose (Calhoun and Swartz, 2005g; Jewett et al., 2008; Swartz, 2006). Mainly enabled by advances from Swartz and colleagues, glucose drives CFPS with a much lower cost and generates more ATP per secondary energy substrate molecule (Calhoun and Swartz, 2005g; Jewett et al., 2008; Swartz, 2006). For example, glucose has a 2:1 molar ratio of secondary energy metabolite to ATP, compared to 1:1 ratio for both CrP and PEP (Kim et al., 2007a). As an extension of the pioneering works above, many groups have turned to use of slowly metabolized glucose polymers to fuel E. coli based CFPS, including starch (Kim et al., 2011), maltodextrin (Caschera and Noireaux, 2015; Wang and Zhang, 2009), and maltose (Caschera and Noireaux, 2014).

While E. coli based CFPS systems have been developed from non-phosphorylated energy substrates, making possible many new applications in industrial biotechnology and rapid prototyping (Bujara et al., 2010; Chappell et al., 2015; Karig et al., 2012; Shin and Noireaux, 2012; Sun et al., 2014; Takahashi et al., 2014; Yin et al., 2012; Zawada et al., 2011), eukaryotic CFPS platforms have been limited to use of high-energy phosphate secondary energy substrates. This includes, for example, a yeast-based CFPS system we developed that leverages creatine phosphate and creatine phosphokinase (CrP/CrK) to power protein synthesis (Choudhury et al., 2014; Gan and Jewett, 2014; Hodgman and Jewett, 2013; Schoborg et al., 2014). The ability to use glucose to fuel CFPS is not only important for CFPS applications, but also can expand the impact of cell-free synthetic biology by joining a rapidly growing number of reports highlighting the ability to co-activate multiple biochemical systems in an integrated cell-free platform (Calhoun and Swartz, 2005a, g; Caschera and Noireaux, 2014, 2015; Fritz et al., 2015; Fritz and Jewett, 2014; Jewett et al., 2008; Jewett et al., 2013; Jewett and Swartz, 2004a, b). As a result, there is a need for improved methods for yeast CFPS that activate natural energy metabolism and avoid the use of expensive high energy phosphate compounds.

### SUMMARY

Disclosed are compositions, methods, and kits for synthesizing biological macromolecules in vitro. The disclosed compositions, methods, and kits may be utilized to perform cell-free protein synthesis, and in particular, cell-free protein synthesis that utilizes natural energy metabolism to improve protein synthesis.

The disclosed compositions may include reaction mixtures for preparing a biological macromolecule in vitro such as a protein. In some embodiments, the disclosed reaction mixture mixtures include: (a) a cell-free extract; (b) a phosphate-free energy source; and (c) a phosphate source. Typically, the reaction mixture does not comprise an exogenous nucleoside triphosphate (e.g., ATP or GTP) or an exogenous nucleoside diphosphate (e.g., ADP or GDP); and/or an exogenous nucleoside triphosphate (e.g., ATP or GTP) or an exogenous nucleoside diphosphate is not added to the reaction mixture, for example, when the reaction mixture is utilized in a cell-free protein synthesis reaction. Optionally, the reaction mixtures may include cAMP and/or cAMP may be added to the reaction mixture, for example, when the reaction mixture is utilized in a cell-free protein synthesis reaction.

The reaction mixtures optionally may include additional components. Optionally, the reaction mixtures may include a buffer.

Optionally, the reaction mixtures may include a translation template (e.g. a translation template encoding a biological macromolecule) and/or a transcription template (e.g., a transcription template that may be transcribed to produce a translation template). Optionally, the reaction mixtures may include a polymerase capable of transcribing a transcription template to form a translation template. Optionally, the reaction mixtures may include one or more nonstandard or non-naturally occurring tRNAs and/or one or more non-standard or non-naturally occurring amino acids (e.g., one or more nonstandard amino acids coupled to a tRNA).

Also disclosed are methods for synthesizing a biological macromolecule in vitro using a cell-free protein synthesis system. The methods typically include synthesizing a biological macromolecule from a translation template in a reaction mixture as described herein, such as a reaction mixture including: (i) a cell-free extract; (ii) a phosphate-free energy source; and (iii) a phosphate source. The disclosed methods may include synthesizing a biological macromolecule from a translation template prepared from a transcription template.

Also disclosed are kits for synthesizing a biological macromolecule in vitro using a cell-free protein synthesis system. The kits may include components for forming a reaction mixture as described herein. In some embodiments, the kits comprise as components: (i) a cell-free extract; (ii) a phosphate-free energy source; and (iii) a phosphate source.

### BRIEF DESCRIPTION OF THE DRAWINGS

FIG. 1. Glycolysis is active in yeast crude extract CFPS. A. Schematic of creatine phosphate (CrP)/creatine kinase (CrK) energy regeneration system. B. Proposed glycolytic energy regeneration system in yeast crude extracts. C. Assessment of glycolytic intermediates to fuel CFPS, six glycolytic intermediates (fructose 1,6-bisphosphate (FBP), phosphoenolpyruvate (PEP), glucose, 3-phosphglyceric acid (3-PGA), pyruvate, and glucose 6-phosphate (G6P)) were added as the sole secondary energy substrate to different yeast CFPS reactions in concentrations ranging from 0 mM to 30 mM and compared to a control composed of no secondary energy substrate (circle). Glucose is the highest yielding non-phosphorylated secondary energy substrate for yeast CFPS. D. Time course reactions of active luciferase for several glycolytic intermediates for equivalent of 30 mM total carbon (e.g., 5 mM glucose or 10 mM PEP). E. HPLC analysis of ethanol production after 4-hour incubation for reactions performed in FIG. 1D. The numbers above each column denote the percentage of theoretical conversion of each secondary energy substrate to ethanol.

FIG. 2. Yeast CFPS CrP/CrK+glucose dual system for energy regeneration does not improve CFPS yields. A. 0 to 25 mM glucose was added to CFPS reactions containing 25 mM creatine phosphate (CrP) and 0.27 mg/mL creatine kinase (CrK). Increasing the starting glucose concentration decreases luciferase yields. B. pH of CFPS reactions containing 25 mM CrP, 0.27 mg/mL CrK, and either 0 mM or 25 mM glucose was measured at regular intervals. C. Assessment of possible ethanol inhibition, various concentrations of ethanol, ranging from 0 mM to 25 mM, were added to CFPS reactions. Active luciferase yields are reported relative to the 0 mM ethanol condition, showing that inhibition was not observed. D. Concentration of ATP was measured at intervals during CFPS reactions including 25 mM CrP, 0.27 mg/mL CrK, and 0 to 25 mM glucose. ATP is rapidly depleted as the starting glucose concentration is increased. Data from panel D traces are individual measurements.

FIG. 3. Optimal starting concentration of glucose. A. Optimal starting concentration of glucose was determined via addition of 0-30 mM of glucose to CFPS reactions containing 0.15 mM cAMP. The optimum was observed at 16 mM glucose. B. Luciferase concentrations measured at regular intervals in CFPS reactions containing 16 mM glucose or 0 mM glucose. C. ATP concentrations measured at regular intervals in CFPS reactions containing 16 mM glucose or 0 mM glucose.

FIG. 4. Optimal amount of exogenous phosphate. A. Optimal amount of exogenous phosphate was determined via addition of 0-50 mM of phosphate to CFPS reactions containing 16 mM glucose. B. Luciferase concentration measured at regular intervals in CFPS reactions containing 16 mM glucose and 25 mM phosphate or 0 mM glucose+0 mM phosphate. C. ATP concentration measured at regular intervals in CFPS reactions containing 16 mM glucose and 25 mM phosphate or 0 mM glucose+0 mM phosphate.

FIG. 5. Optimizing CFPS system with 16 mM glucose. The chemical environment of batch CFPS reactions was optimized by adding varying concentrations of magnesium glutamate (Mg(Glu)₂) and cyclic adenosine monophosphate (cAMP). A. Optimal concentration of Mg(Glu)₂ was extract dependent, but was always between 4 to 6 mM. In this representative plot, the optimal concentration of Mg(Glu)₂ is 5 mM. Luciferase yields are reported relative to the 5 mM Mg(Glu)₂ condition. B. Optimal concentration of cAMP was 0.15 mM. Values shown are means with error bars representing the standard deviation of at least three independent experiments.

FIG. 6. Optimizing yeast CFPS reactions with starch. A. Soluble starch was added to the CFPS reaction in concentrations ranging from 0% to 3% weight starch/volume reaction (w/v). The optimal concentration of starch in the CFPS reactions was 1.4% (w/v). B. Concentrations of luciferase were measured at regular intervals during CFPS reactions with 1.4% (w/v) starch or 0% (w/v) soluble starch. C. Concentrations of ATP were measured at regular intervals during CFPS reactions with 1.4% (w/v) starch or 0% (w/v) soluble starch. D. Varying concentrations of alpha-glucosidase, amyloglucosidase, or no exogenous enzymes were added to CFPS reactions containing 1.4% (w/v) starch. Luciferase yields are reported relative to the 0 μg/mL enzyme condition. Values shown are means with error bars representing the standard deviation of at least three independent experiments.

FIG. 7. Glucose metabolism regenerates energy to fuel protein synthesis. A. The definition of the adenylate energy charge (E.C.) as described by Atkinson (Atkinson, 1968). In vivo studies have shown that energy is limiting when E.C.<0.8 (Chapman et al., 1971). B. Energy charge and luciferase concentration are plotted as a function of reaction time for CFPS reactions containing 16 mM glucose and 25 mM phosphate. The energy charge is >0.8 when protein synthesis begins, between t=2−3 hours. Values shown are means with error bars representing the standard deviation of at least three independent experiments.

FIG. 8. The optimal concentration of cAMP in the glucose CFPS system is not affected by the addition of 25 mM phosphate. The chemical environment of the CFPS reactions with glucose and phosphate was optimized by adding cyclic adenosine monophosphate (cAMP). Values shown are means with error bars representing the standard deviation of at least three independent experiments.

FIG. 9. Glucose and phosphate system achieves improved relative protein yields compared to the state-of-the-art CrP/CrK system. Here we compare the traditional CrP/CrK system to the novel glucose and glucose/phosphate system reported here as measured by active protein synthesis yield (μg/mL; left axis) and relative protein yield (μg protein synthesized per S reagent cost; right axis). Substrate cost includes all substrates used to treat the crude extract, make the genetic template, and assemble the CFPS reaction. Values shown are means with error bars representing the standard deviation of at least three independent experiments.

### DETAILED DESCRIPTION

The present invention is described herein using several definitions, as set forth below and throughout the application.

### Definitions and Terminology

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a phosphate-free energy source” should be interpreted to mean “one or more phosphate-free energy sources” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Polynucleotides and Synthesis Methods

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleotide triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids, which optionally may refer to amino acids other than one or more of alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine residues. The term “amino acid residue” may include alpha-, beta-, gamma-, and delta-amino acids.

In some embodiments, the term “amino acid residue” may include nonstandard, noncanonical, or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard, nancanonical, or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 14ufal4hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≧100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

As used herein, “translation template” refers to an RNA product of transcription from an expression template that can be used by ribosomes to synthesize polypeptides or proteins.

As used herein, coupled transcription/translation (“Tx/Tl”), refers to the de novo synthesis of both RNA and a sequence defined biopolymer from the same extract. For example, coupled transcription/translation of a given sequence defined biopolymer can arise in an extract containing an expression template and a polymerase capable of generating a translation template from the expression template. Coupled transcription/translation can occur using a cognate expression template and polymerase from the organism used to prepare the extract. Coupled transcription/translation can also occur using exogenously-supplied expression template and polymerase from an orthogonal host organism different from the organism used to prepare the extract. In the case of an extract prepared from a yeast organism, an example of an exogenously-supplied expression template includes a translational open reading frame operably coupled a bacteriophage polymerase-specific promoter and an example of the polymerase from an orthogonal host organism includes the corresponding bacteriophage polymerase.

As used herein, Energy Charge (E.C.) refers to the overall status of energy availability in a system (Eq. 1):

$$E . C . = \frac{[ ATP ] + \frac{1}{2} [ ADP ]}{[ ATP ] + [ ADP ] + [ AMP ]} . \qquad ( 1 )$$

Energy Charge can be calculated by initially determining the concentrations of ATP, ADP and AMP in the extract as a function of time during Tx/Tl CFPS reaction. The Energy Charge of a control extract not used in a CFPS reaction can be used a reference state for the initial Energy Charge of a CFPS reaction. Alternatively, Energy Charge for a CFPS reaction can be assessed for a given extract prior to performing CFPS reaction with the extract (e.g., before adding a required reaction component, such as an expression template or a required polymerase).

The term “reaction mixture,” as used herein, refers to a solution containing reagents necessary to carry out a given reaction. A “CFPS reaction mixture” typically contains a crude or partially-purified yeast extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

An aspect of the invention is a platform for preparing a biological macromolecule in vitro. In some embodiments, the biological macromolecule is an oligopeptide or a protein. In certain embodiments, the biological macromolecule is an oligopeptide comprising a nonstandard amino acid subunit or a protein comprising a nonstandard amino acid subunit. The biological macromolecule may be endogenous to yeast, and in specific cases endogenous to the yeast from which a yeast cell-free extract is prepared. In other embodiments the biological macromolecule may be exogenous to yeast or exogenous to the yeast from which a yeast cell-free extract is prepared.

Methods for performing in vitro protein synthesis have been described in published U.S. patent applications, see, e.g., U.S. Published Application Nos. 2015-0259757, 2014-0295492, 2012-0171720, 2008-0138857, 2007-0154983, 2005-0054044, and 2004-0209321. The contents of these published U.S. patent applications is incorporated in the present application by reference in their entireties.

Yeast Cell-Free Protein Synthesis

The platform for preparing a biological macromolecule in vitro comprises a reaction mixture comprising a yeast cell-free extract, a phosphate-free energy source, and a phosphate source. Because CFPS exploits an ensemble of catalytic proteins prepared from the crude lysate of cells, the cell extract (whose composition is sensitive to growth media, lysis method, and processing conditions) is a critical component of extract-based CFPS reactions. A variety of methods exist for preparing an extract competent for cell-free protein synthesis, including U.S. patent application Ser. No. 14/213,390 to Michael C. Jewett et al., entitled METHODS FOR CELL-FREE PROTEIN SYNTHESIS, filed Mar. 14, 2014, and now published as U.S. Patent Application Publication No. 20140295492 on Oct. 2, 2014, which is incorporated by reference.

Yeast extracts for CFPS platforms disclosed herein can be prepared in a variety of ways. Examples of schemes for making yeast extracts are provided in Iizuka et al. (1994) and Iizuka & Sarnow (1997). In another scheme for preparing cellular extract includes three steps: (1) expanding a yeast cell culture in a bioreactor; (2) performing mechanical lysis of the cells by high-pressure homogenization; (3) performing a buffer exchange to generate the resultant extracts for the CFPS platform. Tangential flow filtration can be used to generate the resultant extract, where CFPS platforms are prepared on a large-scale process in industry. In most cases, however, dialysis is preferred in part for ease of use where CFPS platforms are prepared on a smaller-scale process in the laboratory.

Yeast cells used for cell-free translation may be harvested during growth in any exponential phase. In some embodiments, yeast cells for CFPS may be harvested in early-exponential growth phase. When yeast cells are harvested in the early-exponential phase, the yeast cultures may have an OD₆₀₀ of less than 5. In other embodiments, yeast cultures may be harvested during growth at mid-exponential to late-exponential growth phase. When yeast cells are harvested in the mid-exponential to late exponential growth phase may have an OD₆₀₀ from about 6 OD₆₀₀ to about 18 OD₆₀₀. For example, source cells for the yeast extracts disclosed herein can be obtained from mid-exponential to late-exponential batch cultures in the range from about 6 OD₆₀₀ to about 18 OD₆₀₀ or fed-batch cultures harvested in mid-exponential to late-exponential phase. Since the cells are rapidly dividing in this phase, they have a highly active translation machinery. Moreover, from a scaling standpoint, the ability to harvest at a later optical density can allow for larger cell mass recovery per fermentation, thereby leading to a larger volume of total crude extract prepared per fermentation for improved overall system economics. Typically, 1 L of cell culture yields about 6 g of wet cell mass when harvested at 12 OD₆₀₀ compared to ˜1.5 g of wet cell mass when harvest at 3 OD₆₀₀. Subsequently, 1 g of wet cell mass leads to ˜2 mL of crude extract.

Yeast culturing techniques and culture media are well known in the art. Exemplary yeast culture media include YPD media (yeast extract (10 g/1), bacto-peptone (20 g/1; Difco) and dextrose (20 g/1), adjusted to pH5.5) and YPAD media (yeast extract (10 g/1), bacto-peptone (20 g/1; Difco), dextrose (20 g/1) and adenine hemisulfate (30 mg/1), adjusted to pH5.5). For Saccharomyces cerevisiae cellular extracts prepared from the mid-exponential to late-exponential cultures having a range of about 6 OD₆₀₀ to about 18 OD₆₀₀, the yeast cells were cultured in YPAD media. Other yeast culture media, including variations of YPD and YPAD, as well as synthetic dextrose, which is composed of 6.7 g L⁻¹ Yeast Nitrogen Base (YNB) (Sigma-Aldrich, St. Louis, Mo.), 20 g L⁻¹ glucose and 50 mM potassium phosphate buffer, pH 5.5, and its variations, can be used to culture the source Saccharomyces cerevisiae cells for the preparation of the crude yeast extracts for the CFPS systems, platforms and reactions disclosed herein.

Furthermore, a step of adding inorganic phosphate to the growth media can increase protein synthesis capability for extracts generated. Typically, cells can be grown in media containing any source of inorganic phosphate, such as potassium phosphate, sodium phosphate, magnesium phosphate, calcium phosphate, among others, including mixed metal phosphates (for example, sodium potassium phosphate). Concentrations of inorganic phosphate range from about 15 mM to about 250 mM, including about 50 mM, about 75 mM, about 100 mM, about 125 mM and about 150 mM, among other concentrations within this range.

The reaction mixture comprises a phosphate-free energy source. An advantage of the present invention is that use of novel secondary energy substrates for CFPS and these novel secondary energy substrates allow for an increase in the relative yield of biological macromolecules per cost of reagents. The phosphate-free energy source may be any phosphate-free energy source that capable of activating natural energy metabolism. In certain embodiments, the phosphate-free energy source is glucose, a glycolytic intermediate, a polymer comprising a glucose subunit, or any combination thereof. The glycolytic intermediate may include fructose 1,6-bisphosphate (FBP), phosphoenolpyruvate (PEP), glucose, 3-phosphglyceric acid (3-PGA), glucose 6-phosphate (G6P), or any combination thereof. The polymer comprising a glucose subunit may be any naturally occurring or synthetically prepared polymer comprising a glucose subunit. The polymer may be a linear polymer or a branched polymer. The polymer may be any length, including without limitation dimers comprised of two subunits of which at least one is a glucose subunit to long-chain polymers comprised of thousands of subunits of which at least one is a glucose subunit, so long as the polymer is capable of activating natural energy metabolism. Examples of polymers suitable to activate natural energy metabolism include without limitation starch, trehalose, dextran, glycogen, cellulose, amylose, and/or other polymeric carbohydrates. In certain embodiments, the phosphate-free energy source (e.g., glucose or a glycolytic intermediate) is present in the reaction mixture at a concentration of at least about 1 mM, 2 mM, 3 mM, 4 mM, 5 mM, 6 mM, 7 mM, 8 mM, 9 mM, 10 mM, 15 mM, 20 mM, 25 mM, to about 30 mM, including without limitation any concentration range bounded by any two of the foregoing values. In certain embodiments the phosphate-free energy source (e.g., glucose or a glycolytic intermediate) is present in the reaction mixture at a concentration greater than 1 mM, greater than 2 mM, greater than 3 mM, greater than 4 mM, greater than 5 mM, greater than 6 mM, greater than 7 mM, greater than 8 mM greater than 9 mM, or greater than 10 mM and/or at a concentration less than 30 mM, less than 29 mM, less than 28 mM, less than 27 mM, 26 mM, less than 25 mM, less than 24 mM, less than 23 mM, less than 22 mM, less than 21 mM, or less than 20 mM (e.g., within a concentration range bounded by any of these concentration values). In certain embodiments, the phosphate-free energy source (e.g., glucose or a glycolytic intermediate) is present in the reaction mixture at a concentration of at least about 0.1% (w/v), at least about 0.5% (w/v), at least about 1.0% (w/v), at least about 1.5% (w/v), at least about 2.0% (w/v), at least about 2.5% (w/v), at least about 3.0% (w/v), at least about 3.5% (w/v), at least about 4.0% (w/v), at least about 4.5% (w/v), or at least about 5.0% (w/v) including without limitation any concentration ranges including any of the foregoing concentrations as endpoints for the range (e.g., a range of about 0.1% (w/v) to about 5.0% (w/v)).

The reaction mixture also may comprise a phosphate source. An advantage of the present invention is the use of certain phosphate sources that allow for an increase in the relative yield of biological macromolecules per cost of reagents. In certain embodiments the phosphate source comprises exogenous phosphate. In certain embodiments, the exogenous phosphate is present in the reaction mixture at a concentration of from at least about 1 mM, 2 mM, 3 mM, 4 mM, 5 mM, 6 mM, 7 mM, 8 mM, 9 mM, 10 mM, 15 mM, 20 mM, 25 mM, to about 30 mM, including without limitation any concentration range bounded by any two of the foregoing values. In certain embodiments the exogeneous phosphate is present in the reaction mixture at a concentration greater than 1 mM, greater than 2 mM, greater than 3 mM, greater than 4 mM, greater than 5 mM, greater than 6 mM, greater than 7 mM, greater than 8 mM greater than 9 mM, or greater than 10 mM and/or at a concentration less than 30 mM, less than 29 mM, less than 28 mM, less than 27 mM, 26 mM, less than 25 mM, less than 24 mM, less than 23 mM, less than 22 mM, less than 21 mM, or less than 20 mM (e.g., within a concentration range bounded by any of these concentration values).

The reaction mixture may comprise an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the platform may be a coupled transcription/translation (“Tx/Tl”) system where synthesis of translation template and a sequence defined biopolymer from the same cellular extract.

The platform may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The platform may comprise an orthogonal translation system. An orthogonal translation system may comprise one or more orthogonal components that are designed to operate parallel to and/or independent of the organism's orthogonal translation machinery. In certain embodiments, the orthogonal translation system and/or orthogonal components are configured to incorporation of unnatural amino acids. An orthogonal component may be an orthogonal protein or an orthogonal RNA. In certain embodiments, an orthogonal protein may be an orthogonal synthetase. In certain embodiments, the orthogonal RNA may be an orthogonal tRNA or an orthogonal rRNA. An example of an orthogonal rRNA component has been described in Application No. PCT/US2015/033221 to Michael C. Jewett et al., entitled TETHERED RIBOSOMES AND METHODS OF MAKING AND USING THEREOF, filed 29 May 2015, which is incorporated by reference. In certain embodiments, one or more orthogonal components may be prepared in vivo or in vitro by the expression of an oligonucleotide template. The one or more orthogonal components may be expressed from a plasmid present in the genomically recoded organism, expressed from an integration site in the genome of the genetically recoded organism, co-expressed from both a plasmid present in the genomically recoded organism and an integration site in the genome of the genetically recoded organism, express in the in vitro transcription and translation reaction, or added exogenously as a factor (e.g., a orthogonal tRNA or an orthogonal synthetase added to the platform or a reaction mixture).

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts (for examples, S12, S30 and S60 extracts).

The temperature may be any temperature suitable for CFPS. Temperature may be in the general range from about 10° C. to about 40° C., including intermediate specific ranges within this general range, include from about 15° C. to about 35° C., from about 15° C. to about 30° C., from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The CFPS reaction mixture can include a reaction buffer comprising any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others. Concentration ranges having any of these specific concentrations as bounding endpoints also are contemplated herein.

The CFPS reaction mixture can comprise a reaction buffer comprising any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The CFPS reaction mixture can comprise a reaction buffer comprising any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The CFPS reaction mixture can comprise a reaction buffer comprising any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. Concentration ranges having any of these specific concentrations as endpoints also are contemplated herein. In preferred aspects, the concentration of inorganic cations can be within the specific range from about 4 mM to about 9 mM and more preferably, within the range from about 5 mM to about 7 mM.

The CFPS reaction mixture can comprise a reaction buffer comprising any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others. Concentration ranges having any of these specific concentrations as endpoints also are contemplated herein.

CFPS reaction mixtures traditionally include exogenous NTPs (i.e., ATP, GTP, CTP, and UTP). An advantage of the present invention is that the addition of expensive exogenous NTPs may be omitted from a CFPS reaction mixture. Surprisingly reaction mixtures that do not comprise an exogenous NTP allow for CFPS reaction that may have higher relative yields than reaction mixtures that do include an exogenous NTP. In some embodiments of the disclosed CFPS reaction mixtures, for example, which do not include any added exogenous NTPs, the total amount of ATP present in the CFPS reaction mixture is no more than 3.0 mM, 2.5 mM, 2.0 mM, 1.5 mM, 1.0 mM, 0.5 mM, 0.1 mM, 0.05 mM, 0.01 mM, 0.005 mM, or 0.001 mM, or less, for example substantially 0 mM (or within a concentration range bounded by any of these values (e.g., 0-0.05 mM).

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Methods for Activating Natural Energy Metabolism for Improving Yeast Cell-Free Protein Synthesis

Disclosed herein are improved methods for yeast CFPS that activate native energy metabolism. The improved methods use a new energy regeneration system for yeast CFPS that uses glucose and phosphate. This novel approach removes the need for an expensive phosphorylated secondary energy source and avoids inhibitory phosphate accumulation. Although the absolute protein yields may not exceed those previously reported with yeast extract and the CrP/CrK system (e.g., Choudhury et al., 2014), the present invention allows for the surprising increase the relative protein yield per cost of reagents (e.g., μg protein/$ reagents). As a result, the present invention allows for a cost-effective eukaryotic CFPS platform for high throughput protein expression, synthetic biology, and proteomic and structural genomic applications. The applications of the disclosed subject matter include improved expression of protein therapeutics on demand; production of protein libraries; functional genomics studies; and improved method for producing proteins for crystallography studies.

As such, disclosed are compositions, methods, and kits for synthesizing biological macromolecules in vitro. The disclosed compositions and methods may be utilized to perform cell-free protein synthesis, and in particular, cell-free protein synthesis that utilizes natural energy metabolism to improve protein synthesis. The disclosed compositions, methods, and kits may include reaction mixtures for preparing a biological macromolecule in vitro such as a protein. In some embodiments, the disclosed reaction mixture mixtures include: (a) a cell-free extract; (b) a phosphate-free energy source; and (c) a phosphate source. Typically, the reaction mixture does not comprise an exogenous nucleoside triphosphate or an exogenous nucleoside diphosphate.

The disclosed mixtures typically include a cell-free extract. The cell-free extract of the disclosed mixtures may include a yeast cell-free extract. Suitable yeast cell-free extracts may include, but are not limited to cell-free extracts of Saccharomyces spp., including cell-free extracts of Saccharomyces cerevisiae. In some embodiments, yeast cell-free extracts may be prepared from mid-exponential to late-exponential cultures in the range from about 6 OD₆₀₀ to about 18 OD₆₀₀. In some embodiments, the yeast cell extracts may include S30 extract or an S60 extract.

The disclosed reaction mixtures typically include a phosphate-free energy source. Suitable phosphate-free energy sources may include, but are not limited to glucose, a glycolytic intermediate, a polymer comprising a glucose subunit, and any combination thereof. Suitable glycolytic intermediates may include but are not limited to fructose 1,6-bisphosphate (FBP), phosphoenolpyruvate (PEP), glucose, 3-phosphglyceric acid (3-PGA), glucose 6-phosphate (G6P), and any combination thereof. Suitable polymers comprising a glucose subunit may include, but are not limited to starches, dextrans, and combinations thereof.

The disclosed reaction mixtures typically include a phosphate source. The phosphate source typically is an exogenous phosphate source. Suitable phosphate sources may include phosphate salts, including salts of phosphoric acid. Suitable phosphate salts may include, but are not limited to potassium phosphate, magnesium phosphate and ammonium phosphate. In some embodiments, the phosphate source provides a concentration of phosphate in the reaction mixture of about 1 mM to about 30 mM.

Optionally, the reaction mixtures may include cAMP. In reaction mixtures that include cAMP, the cAMP may be present in the reaction mix at a concentration of from about 0.05 mM to about 5 mM.

Optionally, the reaction mixture may include a translation template (e.g., that encodes a biological macromolecule synthesized in the methods disclosed herein), a transcription template (e.g., which may be transcribed to prepare a translation template as disclosed herein), or both a translation template and a transcription template. Optionally, the reaction mixture may include a polymerase capable of transcribing a transcription template to form a translation template (e.g., a DNA-dependent RNA polymerase).

Optionally, the reaction mixture may include a buffer or buffering system. For example, the reaction mixture may include a buffer or buffering system for performing a cell-free protein synthesis reaction.

Optionally, the reaction mixture may include one or more non-standard tRNAs and/or one or more non-standard amino acids. For example, the reaction mixture may include one or more non-standard tRNAs coupled to a non-standard amino acid where the reaction mixture is reacted to produce an oligopeptide or protein comprising the non-standard amino acid.

Also disclosed are methods for synthesizing a biological macromolecule in vitro using a cell-free protein synthesis system. The methods typically include synthesizing a biological macromolecule from a translation template in a reaction mixture as described herein, such as a reaction mixture including: (i) a cell-free extract (e.g., a yeast cell-free extract as disclosed herein); (ii) a phosphate-free energy source (e.g., glucose, a glycolytic precursor, a polymer comprising glucose as a monomer, or a combination thereof as disclosed herein); and (iii) a phosphate source (e.g., a phosphate salt as disclosed herein). In the methods, the translation template may be transcribed from a DNA template. The disclosed methods may be performed to synthesize biological macromolecules as a batch reaction or as a continuous reaction.

Also disclosed are kits for synthesizing a biological macromolecule in vitro using a cell-free protein synthesis system. The kits may include components for forming a reaction mixture as described herein. In some embodiments, the kits comprise as components: (i) a cell-free extract (e.g., a yeast cell-free extract as disclosed herein); (ii) a phosphate-free energy source (e.g., glucose, a glycolytic precursor, a polymer comprising glucose as a monomer, or a combination thereof as disclosed herein); and (iii) a phosphate source (e.g., a phosphate salt as disclosed herein).

The disclosed methods and/or compositions may be practiced and/or prepared by using and/or modifying methods and/or compositions in the art. (See, e.g., Anderson et al., “Energizing eukaryotic cell-free protein synthesis with glucose metabolism,” FEBS Lett. 2015 Jul. 8; 589(15):1723-7; Hodgman et al., “Characterizing IGR IRES-mediated translation initiation for use in yeast cell-free protein synthesis,” N Biotechnol. 2014 Sep. 25; 31(5):499-505; Schoborg et al., “Substrate replenishment and byproduct re oval improve yeast cell-free protein synthesis,” Biotechnol J. 2014 May; 9(5):630-40; Hodgman et al., “Optimized extract preparation methods and reaction conditions for improved yeast cell-free protein synthesis,” Biotechnol Bioeng. 2013 October; 110(10):2643-54; Carlson et al., “Cell-free protein synthesis: applications come of age,” Biotechnol Adv. 2012 Sep.-Oct.; 30(5):1185-94; and Hodgman et al., “Cell-free synthetic biology: thinking outside the cell,” Metab Eng. 2012 May; 14(3):261-9; Perez et al., “Cell-Free Synthetic Biology: Engineering Beyond the Cell, Cold Spring Harb Perspect Biol. 2016 Dec. 1; 8(12); the contents of which are incorporated herein by reference in their entireties.

### ILLUSTRATIVE EMBODIMENTS

The following embodiments are illustrative and are not intended to limit the scope of the claimed subject matter.

### Embodiment 1

A reaction mixture for preparing a biological macromolecule in vitro, the reaction mixture comprising: (a) a yeast cell-free extract; (b) a phosphate-free energy source; and (c) a phosphate source.

### Embodiments 2

The reaction mixture of embodiment 1, wherein said phosphate-free energy source is selected from a group consisting of glucose, a glycolytic intermediate, a polymer comprising a glucose subunit, and any combination thereof.

### Embodiment 3

The reaction mixture of embodiment 1 or 2, wherein the polymer comprising a glucose subunit is starch or starch, trehalose, dextran, glycogen, cellulose, amylose, and/or other polymeric carbohydrates.

### Embodiment 4

The reaction mixture of embodiment 2, wherein the glycolytic intermediate is selected from the group consisting of fructose 1,6-bisphosphate (FBP), phosphoenolpyruvate (PEP), glucose, 3-phosphglyceric acid (3-PGA), glucose 6-phosphate (G6P), and any combination thereof.

### Embodiment 5

The reaction mixture of any of the foregoing embodiments, wherein the phosphate source comprises exogenous phosphate.

### Embodiment 6

The reaction mixture of embodiment 5, wherein exogenous phosphate is present in the reaction mixture at a concentration of from about 1 mM to about 30 mM.

### Embodiment 7

The reaction mixture of embodiment 5, wherein exogenous phosphate is selected from a group consisting of potassium phosphate, magnesium phosphate and ammonium phosphate.

### Embodiment 8

The reaction mixture of any of the foregoing embodiments further comprising cAMP.

### Embodiment 9

The reaction mixture of embodiment 8, wherein cAMP is present in the reaction mix at a concentration of from about 0.05 mM to about 5 mM.

### Embodiment 10

The reaction mixture of any of the foregoing embodiments, wherein the yeast cell-free extract is a Saccharomyces cerevisiae cell-free extract.

### Embodiment 11

The reaction mixture of any of the foregoing embodiments, wherein the yeast cell-free extract is prepared from mid-exponential to late-exponential culture in the range from about 6 OD₆₀₀ to about 18 OD₆₀₀.

### Embodiment 12

The reaction mixture of any of the foregoing embodiments, wherein the yeast cell-free extract is an S30 extract or an S60 extract.

### Embodiment 13

The reaction mixture of any of the foregoing embodiments further comprising a reaction buffer.

### Embodiment 14

The reaction mixture of any of the foregoing embodiments further comprising a translation template, a transcription template, or both a translation template and a transcription template.

### Embodiment 15

The reaction mixture of any of the foregoing embodiments further comprising a polymerase capable of transcribing a transcription template to form a translation template.

### Embodiment 16

The reaction mixture of any of the foregoing embodiments, wherein the reaction mixture does not comprise an exogenous nucleoside triphosphate.

### Embodiment 17

The reaction mixture of any of the foregoing embodiments, wherein the biological macromolecule is an oligopeptide or a protein.

### Embodiment 18

The reaction mixture of embodiment 17, wherein the biological macromolecule is an oligopeptide comprising a nonstandard amino acid subunit or a protein comprising a nonstandard amino acid subunit.

### Embodiment 19

A method for synthesis of a biological macromolecule in vitro using yeast cell-free protein synthesis, comprising: (a) synthesizing the biological macromolecule from a translation template in a reaction mixture comprising: (i) a yeast cell-free extract; (ii) a phosphate-free energy source; and (iii) a phosphate source.

### Embodiment 20

The method of embodiment 19, wherein the reaction mixture is the reaction mixture of embodiment 1.

### Embodiment 21

The method of embodiment 19 or 20, wherein the translation template is transcribed from a DNA template.

### Embodiment 22

The method of any of embodiments 19-21, wherein said synthesis of biological macromolecules is performed as a batch reaction.

### Embodiment 23

The method of any of embodiments 19-22, wherein said synthesis of biological macromolecules is performed as a continuous reaction.

### Embodiment 24

The method of any of embodiments 19-23, wherein the biological macromolecule is an oligopeptide or a protein.

### Embodiment 25

The method of any of embodiments 19-24, wherein the biological macromolecule is an oligopeptide comprising a nonstandard amino acid subunit or a protein comprising a nonstandard amino acid subunit.

### Embodiment 26

A kit comprising any components of the reaction mixtures of embodiments 1-18 and/or any components of the methods of embodiments 19-25.

### EXAMPLES

The following Examples are illustrative and are not intended to limit the scope of the claimed subject matter. Reference is made to Anderson et al., “Energizing eukaryotic cell-free protein synthesis with glucose metabolism,” FEBS Letters, Jul. 8, 2015, Volume 589, Issue 15, Pages 1723-1727, the content of which is incorporated herein by reference in its entirety.

Energizing Eukaryotic Cell-Free Protein Synthesis with Glucose Metabolism

Abstract

Cell-free protein synthesis (CFPS) is a powerful technology with a growing number of applications, including high-throughput synthesis of protein libraries and characterization of genetic components. There currently exist high-yielding Escherichia coli CFPS platforms that avoid the use of expensive high-energy phosphate compounds. However, eukaryotic CFPS systems to date generally utilize such compounds to regenerate the adenosine triphosphate (ATP) necessary to drive protein synthesis. Expensive reagent costs, among other issues have prevented the widespread use and practical implementation of eukaryotic CFPS technology. In this study, we report the development of the first ever eukaryotic CFPS system powered by natural energy metabolism of non-phosphorylated energy substrates, to our knowledge. To achieve this, we screened six different glycolytic intermediates for their ability to regenerate ATP and fuel protein synthesis in a Saccharomyces cerevisiae crude extract CFPS platform. We observed the synthesis of 1.05±0.12 μg mL⁻¹ active luciferase when using 16 mM glucose as a secondary energy substrate and demonstrated that glycolysis is active by quantifying the production of ethanol during the reaction. With the addition of 25 mM potassium phosphate, our yields using glucose increased approximately 3.5-fold to 3.64±0.35 μg mL⁻¹. The increase in protein yield is shown to be due to the prolonged availability of ATP. Although synthesis yields on a gram per liter basis remain lower than the CrP/CrK system previously developed, the relative protein yield (μg protein/$ reagents) has increased by 16%. This work provides the first evidence that glycolytic metabolism is active in eukaryotic crude extract CFPS platforms and represents the first eukaryotic CFPS platform powered by non-phosphorylated energy substrates. This demonstration provides the foundation for development of cost-effective eukaryotic CFPS platforms from multiple host organisms for high-throughput protein expression, synthetic biology, and proteomic and structural genomic applications.

Introduction

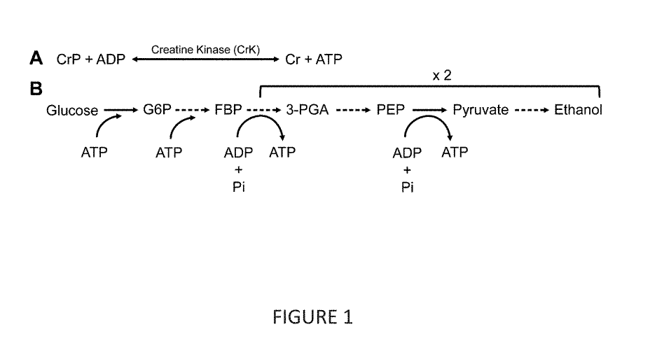

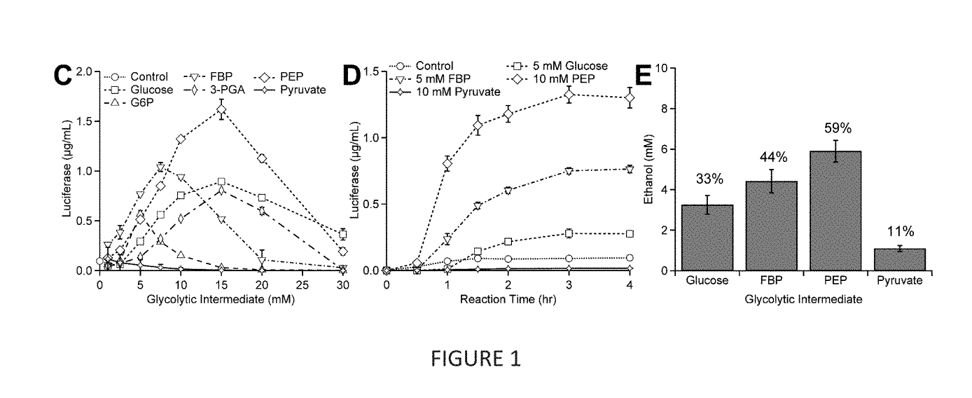

Cell-free protein synthesis (CFPS) is an emerging field that allows for the synthesis of proteins without maintaining the necessary requirements for cell growth (Carlson et al., 2012). One of the major limitations of CFPS is the high cost of reagents, with phosphorylated energy compounds accounting for the bulk of the reaction cost (Calhoun and Swartz, 2005a). At present, the most commonly used secondary energy sources for CFPS contain high-energy phosphate bonds such as creatine phosphate (CrP) used in all eukaryotic CFPS platforms (Brödel et al., 2013; Hodgman and Jewett, 2013a; Takai et al., 2010) and phosphoenolpyruvate (PEP) used in many bacterial CFPS platforms (Kim and Swartz, 2001). Additional cost is added to the reaction if exogenous enzymes are required to catalyze ATP regeneration (e.g. creatine phosphokinase (CrK)) (FIG. 1A).

Within the last decade, the E. coli CFPS platform has overcome some cost limitations by replacing PEP with glucose. Using a non-phosphorylated energy source also allows for the recycling of inorganic phosphate to synthesize ATP (Caschera and Noireaux, 2014; Wang and Zhang, 2009), which has been previously shown to be inhibitory to E. coli (Kim and Swartz, 2000) and yeast (Schoborg et al., 2013) CFPS reactions. Furthermore, glucose has a 2:1 molar ratio of secondary energy metabolite to ATP (FIG. 1B), compared to 1:1 ratio for both CrP and PEP (Kim et al., 2007a).

However, there are limitations associated with using glucose as the secondary energy source in bacterial CFPS reactions. One known limitation is that glucose is rapidly metabolized, resulting in accumulation of lactate and acetate and changes in the reaction pH, which inhibit protein synthesis (Calhoun and Swartz, 2005b). To overcome this limitation, many groups have turned to slowly metabolized glucose polymers including starch (Kim et al., 2011), maltodextrin (Wang and Zhang, 2009), and maltose (Caschera and Noireaux, 2014).

At present, all eukaryotic CFPS platforms require creatine phosphate and creatine phosphokinase (CrP/CrK) to power protein synthesis, including a yeast-based system previously developed in our lab (Choudhury et al., 2014; Hodgman and Jewett, 2013b; Schoborg et al., 2013). In this study, we demonstrate that it is possible to power yeast CFPS reactions with non-phosphorylated energy sources and have reached synthesis yields of 1.05±0.12 μg mL⁻¹ active luciferase with 16 mM glucose. Ultimately, we optimized our glucose energy system with the addition of cyclic AMP (cAMP) and exogenous phosphate, reaching yields of 3.64±0.35 μg mL⁻¹ active luciferase. To the best of our knowledge, our work is the first example of powering a eukaryotic CFPS reaction from the native glycolytic pathway.

Materials and Methods

Yeast extract preparation, CFPS reactions, and luciferase quantification were performed as previously described (Choudhury et al., 2014; Hodgman and Jewett, 2013; Schoborg et al., 2014), with the exception the energy regeneration system (CrP/CrK) was replaced with glycolytic intermediates.

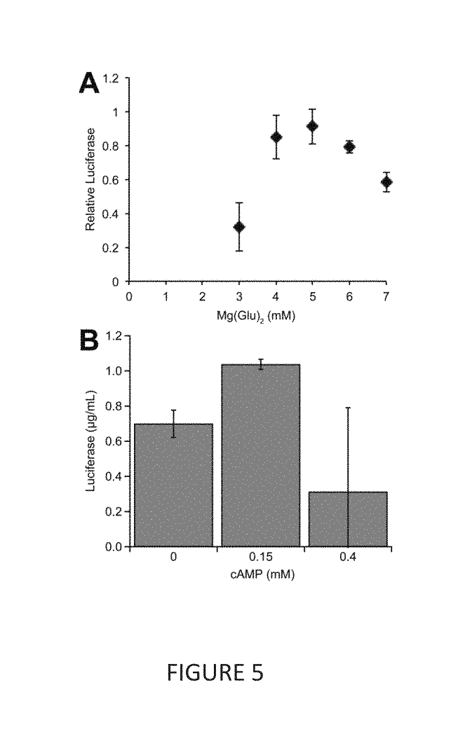

The concentration of magnesium glutamate (Mg(Glu)₂) added to CFPS reactions was optimized for each extract, as CFPS yields are known to be sensitive to magnesium (Hodgman and Jewett, 2013) (e.g., FIG. 5). We tested glucose, glucose-6-phosphate (G6P), 3-phosphoglyceric acid (3-PGA), phosphoenolpyruvate (PEP), fructose-1,6-bisphosphate (FBP), and pyruvate in concentrations ranging from 0-30 mM. We also tested CFPS reactions containing glucose in concentrations ranging from 0-25 mM glucose in combination with the CrP/CrK energy regeneration system. When denoted, 0.15 mM cAMP and phosphate (in the form of potassium phosphate, pH 7.4) were included in the reaction mixture. In reactions containing potassium phosphate, the overall potassium concentration is balanced by reducing the concentration of potassium glutamate. Reaction conditions can be found in Table 1.

TABLE 1
Final concentration of components used for CrP/CrK- and
glucose-powered CFPS systems.

| | CrP/Crk | | Glucose | |
|---|---|---|---|---|
| Reagents | System | | System | |
| Salts and polyamines: | | | | |
| Magnesium glutamate (Mg(Glu)₂) | 4-6 | mM | 4-6 | mM |
| Potassium glutamate (KGlu) | 120 | mM | 120 | mM |
| Spermidine | 0.50 | mM | 0.50 | mM |
| Putrescine | 2 | mM | 2 | mM |
| NTPs (ATP, GTP, UTP, and CTP) | 1.50 | mM | 1.50 | mM |
| [individual concentration] | | | | |
| 20 amino acids [individual | 80 | μM | 80 | μM |
| concentration] | | | | |
| DTT | 1.2 | mM | 4 | mM |
| Creatine Phosphate | 25 | mM | 0 | mM |
| Creatine Phosphokinase | 0.27 | mg/mL | 0 | mg/mL |
| Transcriptional and translational | | | | |
| components: | | | | |
| Yeast Extract | 2.80 | mg/mL | 2.80 | mg/mL |
| Reporter PCR Template: ΩLucA₅₀ | 6.67 | μg/mL | 6.67 | μg/mL |
| T7 RNA polymerase | 0.027 | mg/mL | 0.027 | mg/mL |
| Other components: | | | | |
| HEPES-KOH, pH 7.6 (total in reaction) | 22 | mM | 22 | mM |
| Glucose | 0 | mM | 16 | mM |
| Phosphate (Potassium Phosphate) | 0 | mM | 25 | mM |
| Glycerol | 11% | | 11% | |
| Cyclic AMP (cAMP) | 0 | mM | 0.15 | mM |

Table 1 illustrates the final concentration of components used for CrP/CrK- and glucose-powered CFPS systems. These values do not include the concentrations of small molecules in the yeast extract. Notably, optimal magnesium glutamate concentrations depend heavily on the amount of magnesium in the extract. Each extract is tested individually to determine optimal [Mg(Glu)₂] as a part of initial studies.

HPLC analysis of ethanol was performed as previously described (Choudhury et al., 2014). Nucleotide analysis was performed as previously described (Schoborg et al., 2014) except the gradient for buffer B was adjusted to: 0 min, 0%; 10 min, 30%; 50 min, 80%; 55 min, 100%; 60 min, end.

Results

Screening of Glycolytic Intermediates.

Six different glycolytic intermediates to fuel combined transcription and translation were screened in 15 μL batch CFPS reactions for 4 h at 21° C. (FIG. 1C). The six intermediates included fructose 1,6-bisphosphate (FBP), phosphoenolpyruvate (PEP), glucose, 3-phosphglyceric acid (3-PGA), pyruvate, and glucose 6-phosphate (G6P) at concentrations ranging from 0-30 mM. The CFPS reaction was programmed to synthesize luciferase as a model reporter protein and combined transcription and translation was enabled by the use of the Ω cap-independent translation initiation leader sequence (Gan and Jewett, 2014). Strikingly, our results demonstrated that it is indeed possible to activate yeast CFPS reactions from glycolytic intermediates upstream of pyruvate, reaching 1.04±0.45 and 1.62±0.10 μg mL⁻¹ when powering the reaction with fructose 1,6-bisphosphate (FBP) and PEP, respectively. Of the six glycolytic intermediates, only pyruvate was unable to function as a secondary energy source (FIG. 1C). The inability of pyruvate to power CFPS was expected due to the lack of ATP regenerating power of pyruvate alone in fermentation metabolic processes.

Time course reactions. Time course CFPS reactions with the three highest-yielding intermediates (FBP, glucose, and PEP) were performed. This revealed that the choice of glycolytic intermediate impacted the rate of protein synthesis but not the reaction duration; in all cases protein synthesis had terminated after 4 hours (FIG. 1D). Negative control reactions performed with pyruvate or no secondary energy substrate produced little to no luciferase (FIG. 1D). The carbon from the glycolytic intermediates is expected to produce ethanol through fermentation, as has been shown in previous works (Buchner and Rapp, 1897; Khattak et al., 2014). Thus, we measured ethanol production to confirm glycolysis was active for each carbon source. As expected, we found that ethanol is synthesized when glucose, 1-BP, and PEP are able to power protein synthesis (FIG. 1E). Ethanol is also produced in the presence of pyruvate, but no protein is synthesized due to limited ATP availability as described above (FIG. 1E).

Dual Glucose-CrP/CrK System.

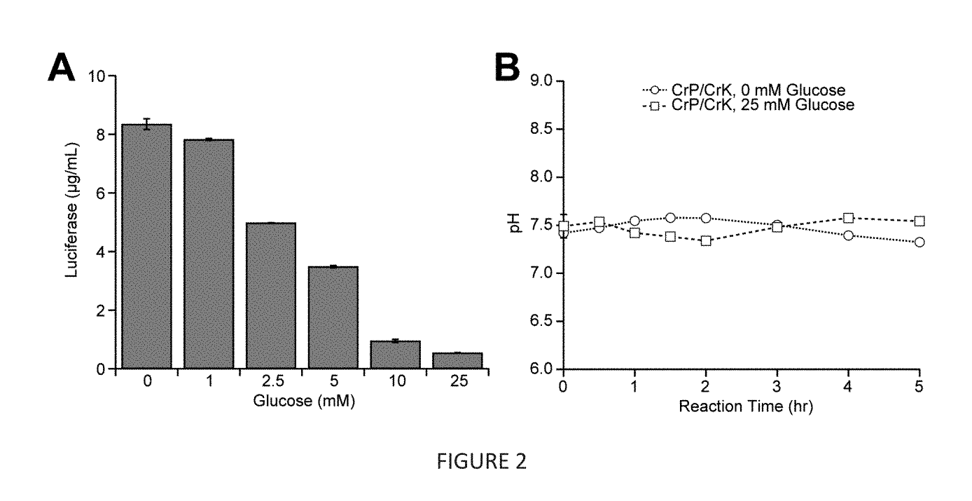

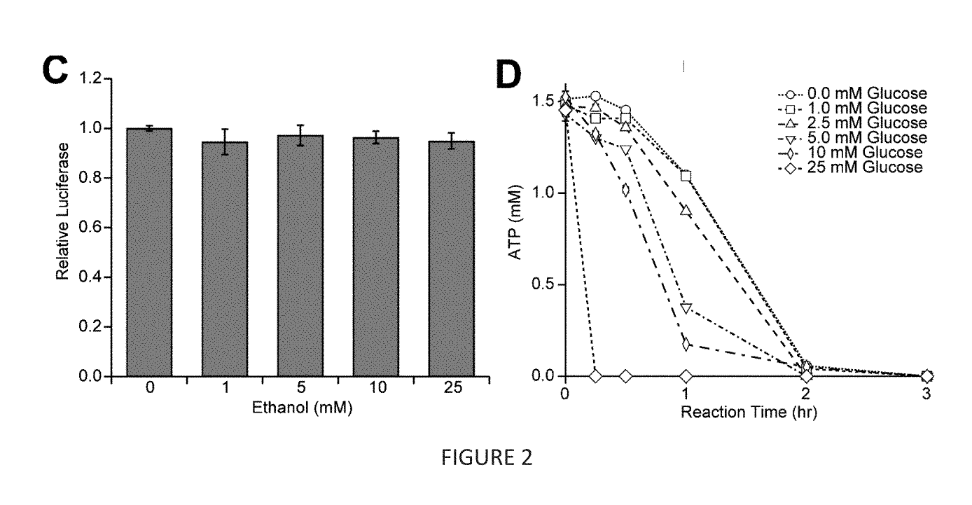

With the goal of increasing protein synthesis yields, we next tested a dual system, in which glucose is used in combination with CrP/CrK. Previously, such a system was demonstrated by Kim et al. to enhance yields in an E. coli CFPS platform (Kim et al., 2007b). Unexpectedly, we found that the addition of glucose to the CrP/CrK system severely inhibits CFPS, with 10 mM glucose addition resulting in an 89% reduction in protein synthesis (FIG. 2A). We reasoned that this could result from a decrease in pH, as seen previously in E. coli CFPS platforms powered by glucose, or a toxicity effect from ethanol accumulation (Calhoun and Swartz, 2005a). However, we observed no change in pH during the course of the reaction (FIG. 2B), and showed that ethanol is not toxic in our reactions at concentrations of up to 25 mM (FIG. 2C), which far exceeded the expected ethanol produced (FIG. 1E). Historically, nonproductive energy consumption has been identified as one of the primary reasons for early termination of CFPS. Thus, we used quantitative HPLC analysis to track the ATP pool over time. Nucleotide analysis revealed that the decrease in protein synthesis yields when glucose is added to the reaction is due to rapid ATP consumption. For example, in the presence of 25 mM glucose, ATP is fully consumed within the first 15 minutes of reaction (FIG. 2D), constraining the ability to produce protein.

Optimization.

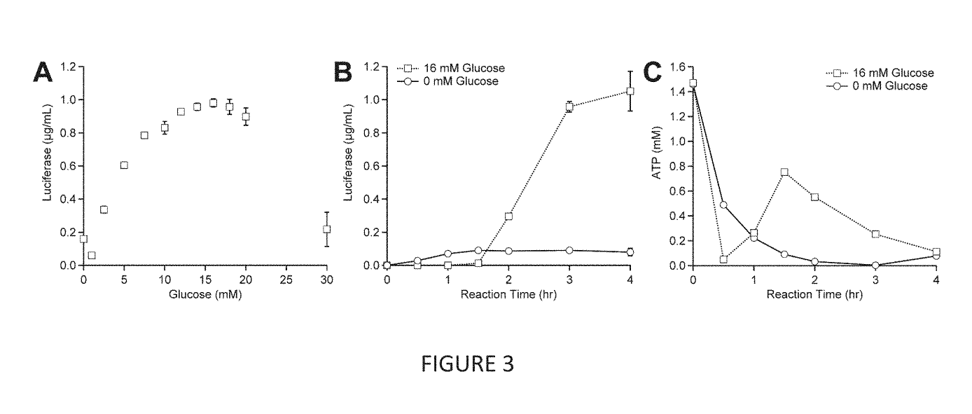

Given the inability to activate a dual energy regeneration system, we returned to the glucose-only system, and determined through an initial optimization that 16 mM glucose is the optimal substrate concentration (FIG. 3A). We subsequently carried out a series of additional optimization experiments to try to increase CFPS. We explored the effects of reaction temperature, magnesium glutamate (Mg(Glu)₂) concentration, potassium glutamate concentration, spermidine concentration and additives such as cyclic AMP (cAMP) (See Table 2).

TABLE 2
Parameters optimized during development of CFPS platforms
powered by glucose metabolism

| | Conditions | | Optimal | |
|---|---|---|---|---|
| Parameter | Tested | | Condition | |
| Magnesium glutamate (Mg(Glu)₂) | 3-7 | mM | 4-6 | mM |
| Potassium glutamate (KGlu) | 80-160 | mM | 120 | mM |
| Spermidine (Spe) | 0-2 | mM | 0.50 | mM |
| Cyclic AMP (cAMP) | 0-0.4 | mM | 0.15 | mM |
| Reaction temperature | 21-30° | C. | 21° | C. |

Table 2 illustrates the parameters optimized during development of CFPS platforms powered by glucose metabolism. These values do not include the concentration of small molecules in the yeast extract. The optimal values for each parameter (right column) were used in all subsequent reactions. (See Table 1).

Despite a rigorous search, we only observed that addition of cAMP increased yields, suggesting that our original conditions for yeast CFPS captured a maximum. The addition of 0.15 mM cAMP increased our yields 1.5-fold, bringing our yields to approximately 1 μg mL⁻¹ (FIG. 5B). The kinetics of protein synthesis follows an interesting trajectory when using glucose and cAMP. Specifically, protein synthesis is delayed when using glucose as the energy source (FIG. 3B), which we attribute to ATP availability. ATP is rapidly consumed in the first 30 minutes of the reaction, but more than 50% is regenerated after 90 minutes (FIG. 3C).

Glucose-Polymer Metabolism.

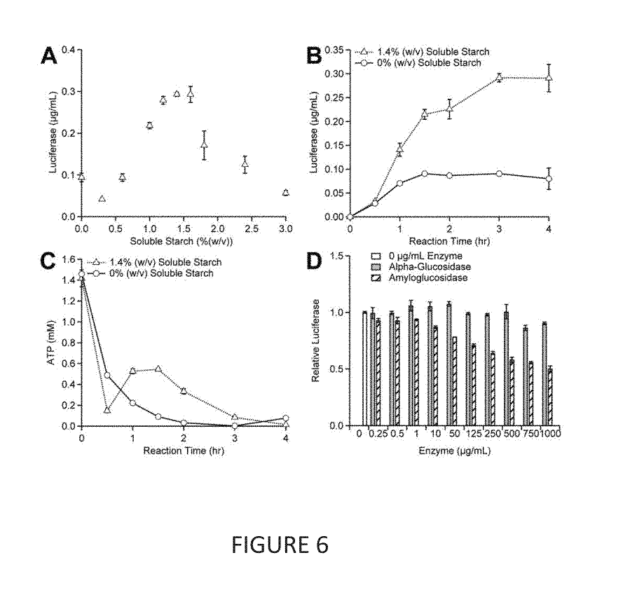

We next investigated the use of slowly metabolized carbon polymers to slow the initial consumption of ATP. We demonstrated that soluble starch can fuel CFPS reaching ˜0.3 μg mL⁻¹ with 1.4% (w/v) starch (FIGS. 6A and 6B). Using starch did not reduce initial consumption of ATP, with only 0.2 mM left after 30 minutes of the reaction (FIG. 6C). Our data suggest that ATP regeneration limits the use of starch when compared to glucose alone. Specifically, the regeneration of ATP when using starch is lower than with 16 mM glucose, leading to a lower protein yield. Supplying a-Glucosidase and amyloglucosidase enzymes did not improve protein synthesis yields, suggesting the activity of our crude lysates is sufficient to metabolize starch (FIG. 6D).

Addition of Inorganic Phosphate.

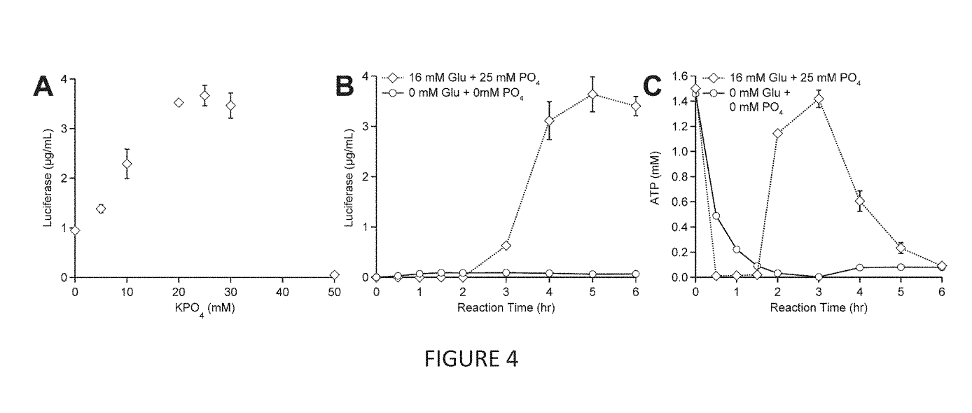

We evaluated the addition of 0-50 mM inorganic phosphate in the form of potassium phosphate to our glucose-driven yeast CFPS system, while keeping the total potassium concentration constant (i.e., addition of potassium phosphate was balanced by adjusting the concentration of potassium glutamate) (FIG. 4A). With the addition of 25 mM inorganic phosphate, CFPS yields increased almost 3.5-fold, reaching 3.64±0.35 μg mL⁻¹ (FIG. 4A). FIG. 4B shows luciferase accumulation over time.

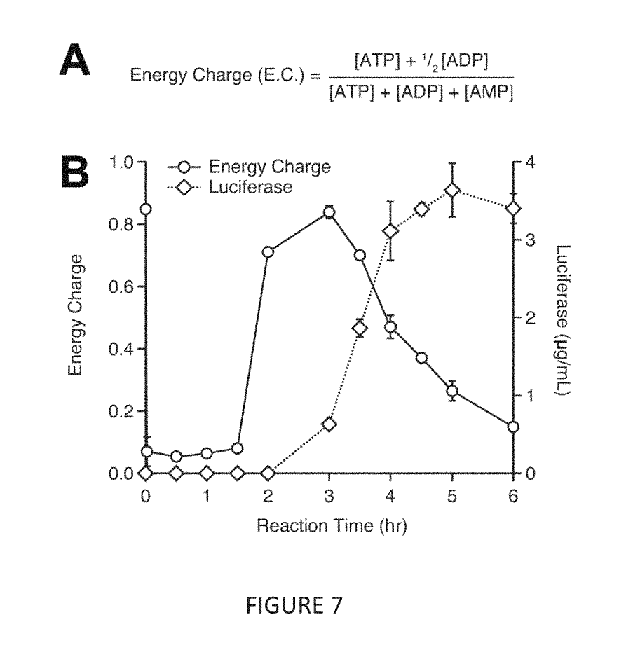

As reported for the glucose and starch systems, protein production appears to be linked to ATP availability, which can be described by Atkinson's Energy Charge (E.C.) calculation (Atkinson, 1968) (FIG. 7A). In vivo studies have shown energy is limiting in systems with an E.C. less than 0.8 (Chapman et al., 1971). In reactions containing glucose and phosphate, we observed that ATP is rapidly consumed within the first 30 minutes of the reaction, but now almost 100% is regenerated after 3 hours (FIG. 4C), enabling protein synthesis to extend to 5 h (FIG. 4B). The observed ATP regeneration coincides exactly with initiation of protein synthesis and the point at which E.C. rises above 0.8, between 2-3 hours (FIG. 7B). Based on our observations from energy charge calculations, we propose that this trend in ATP concentration is observed due to the activation of glucose metabolism. At the start of the reaction, ATP is consumed in the pay-in phase of glycolysis while glucose is metabolized. After all available glucose has been consumed, ATP is regenerated by glucose metabolism and accumulates until sufficient ATP is available for protein synthesis.

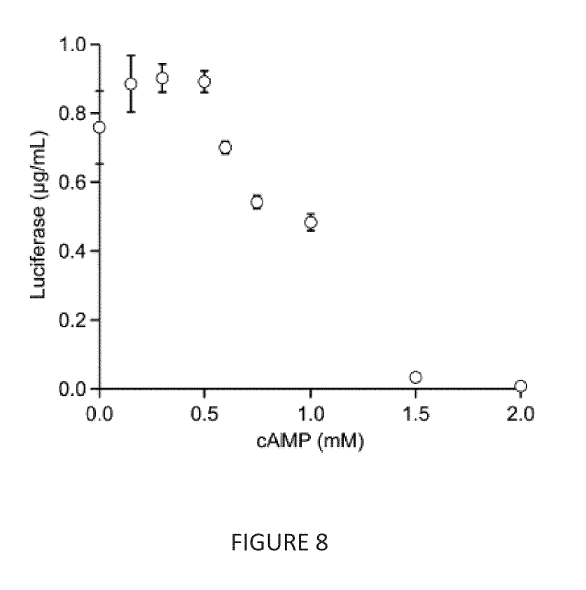

As compared to the glucose only system, ATP regeneration is improved in the glucose/phosphate system, resulting in prolonged availability of a high concentration of ATP, which manifests in higher protein synthesis yields. This is the longest reported batch yeast CFPS reaction to date, to the best of our knowledge. In follow-up experiments, we confirmed that the optimal concentrations of cAMP remained the same in the glucose/phosphate energy system as in the glucose system (FIG. 8).

Summary

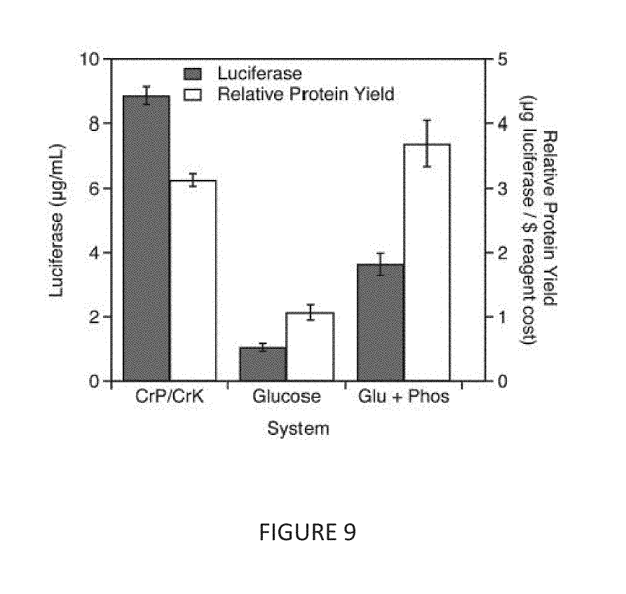

In summary, we have developed a new energy regeneration system with glucose and phosphate that removes the need for an expensive phosphorylated secondary energy source to power yeast CFPS. To our knowledge, this is the first time that a eukaryotic-based CFPS system has been powered by natural energy metabolism of a non-phosphorylated energy substrate. Although our yields do not exceed those previously reported with yeast extract and the CrP/CrK system (Choudhury et al., 2014), we have increased the relative protein yield (μg protein/$ reagents) by 16% with our novel glucose/phosphate system (FIG. 9). Further optimization of this platform through host strain engineering or the use of nucleotide monophosphates, as has been done in E. coli-based systems (Calhoun and Swartz, 2005a), will result in a cost-effective eukaryotic CFPS platform for high throughput protein expression, synthetic biology, and proteomic and structural genomic applications.

### REFERENCES

Atkinson, D. E., (1968) The energy charge of the adenylate pool as a regulatory parameter. Interaction with feedback modifiers. Biochemistry 7, 4030-4034.
Brödel, A. K., Sonnabend, A., Roberts, L. O., Stech, M., Wüstenhagen, D. A., Kubick, S., (2013) IRES-Mediated Translation of Membrane Proteins and Glycoproteins in Eukaryotic Cell-Free Systems. PLoS ONE 8, e82234.
Buchner, E., Rapp, R., (1897) Alkoholische gährung ohne hefezellen. Berichte der deutschen chemischen Gesellschaft 30, 2668-2678.
Bujara, M., Schumperli, M., Billerbeck, S., Heinemann, M., Panke, S., (2010) Exploiting cell-free systems: Implementation and debugging of a system of biotransformations. Biotechnol Bioeng 106, 376-389.
Calhoun, K. A., Swartz, J. R., (2005a) An economical method for cell-free protein synthesis using glucose and nucleoside monophosphates. Biotechnology progress 21, 1146-1153.
Calhoun, K. A., Swartz, J. R., (2005g) Energizing cell-free protein synthesis with glucose metabolism. Biotechnology and bioengineering 90, 606-613.
Carlson, E. D., Gan, R., Hodgman, C. E., Jewett, M. C., (2012) Cell-free protein synthesis: applications come of age. Biotechnology advances 30, 1185-1194.
Caschera, F., Noireaux, V., (2014) Synthesis of 2.3 mg/ml of protein with an all Escherichia coli cell-free transcription-translation system. Biochimie 99, 162-168.
Caschera, F., Noireaux, V., (2015) A cost-effective polyphosphate-based metabolism fuels an all E. coli cell-free expression system. Metabolic engineering 27, 29-37.
Chapman, A. G., Fall, L., Atkinson, D. E., (1971) Adenylate energy charge in Escherichia coli during growth and starvation. J Bacteriol 108, 1072-1086.
Chappell, J., Takahashi, M. K., Lucks, J. B., (2015) Creating small transcription activating RNAs. Nature chemical biology 11, 214-220.
Choudhury, A., Hodgman, C. E., Anderson, M. J., Jewett, M. C., (2014) Evaluating fermentation effects on cell growth and crude extract metabolic activity for improved yeast cell-free protein synthesis. Biochemical Engineering Journal 91, 140-148.
Fritz, B. R., Jamil, O. K., Jewett, M. C., (2015) Implications of macromolecular crowding and reducing conditions for in vitro ribosome construction. Nucleic acids research.
Fritz, B. R., Jewett, M. C., (2014) The impact of transcriptional tuning on in vitro integrated rRNA transcription and ribosome construction. Nucleic acids research 42, 6774-6785.
Gan, R., Jewett, M. C., (2014) A combined cell-free transcription-translation system from Saccharomyces cerevisiae for rapid and robust protein synthesis. Biotechnology journal 9, 641-651.
Hodgman, C. E., Jewett, M. C., (2012) Cell-free synthetic biology: thinking outside the cell. Metabolic engineering 14, 261-269.
Hodgman, C. E., Jewett, M. C., (2013) Optimized extract preparation methods and reaction conditions for improved yeast cell-free protein synthesis. Biotechnology and bioengineering 110, 2643-2654.
Hong, S. H., Kwon, Y. C., Martin, R. W., Des Soye, B. J., de Paz, A. M., Swonger, K. N., Ntai, I., Kelleher, N. L., Jewett, M. C., (2015) Improving Cell-Free Protein Synthesis through Genome Engineering of Escherichia coli Lacking Release Factor 1. Chembiochem: a European journal of chemical biology 16, 844-853.
Iizuka, N. et al. (1994) Cap-dependent and cap-independent translation by internal initiation of mRNAs in cell extracts prepared from Saccharomyces cerevisiae. Mol. Cell. Biol. 14, 7322.
Iizuka, N. & Sarnow, P. (1997) Translation-competent extracts from Saccharomyces cerevisiae: Effects of L-A RNA, 5′ cap, and 3′ poly(A) tail on translational efficiency of mRNAs. Methods 11, 353.
Jewett, M. C., Calhoun, K. A., Voloshin, A., Wuu, J. J., Swartz, J. R., (2008) An integrated cell-free metabolic platform for protein production and synthetic biology. Molecular systems biology 4, 220.
Jewett, M. C., Fritz, B. R., Timmerman, L. E., Church, G. M., (2013) In vitro integration of ribosomal RNA synthesis, ribosome assembly, and translation. Molecular systems biology 9, 678.
Jewett, M. C., Swartz, J. R., (2004a) Mimicking the Escherichia coli cytoplasmic environment activates long-lived and efficient cell-free protein synthesis. Biotechnology and bioengineering 86, 19-26.
Jewett, M. C., Swartz, J. R., (2004b) Substrate replenishment extends protein synthesis with an in vitro translation system designed to mimic the cytoplasm. Biotechnology and bioengineering 87, 465-472.
Karig, D. K., Iyer, S., Simpson, M. L., Doktycz, M. J., (2012) Expression optimization and synthetic gene networks in cell-free systems. Nucleic Acids Res 40, 3763-3774.
Khattak, W. A., Ul-Islam, M., Ullah, M. W., Yu, B., Khan, S., Park, J. K., (2014) Yeast cell-free enzyme system for bio-ethanol production at elevated temperatures. Process Biochemistry 49, 357-364.
Kim, D.-M., Swartz, J. R., (2000) Prolonging cell-free protein synthesis by selective reagent additions. Biotechnol Frog 16, 385-390.
Kim, D. M., Swartz, J. R., (2001) Regeneration of adenosine triphosphate from glycolytic intermediates for cell-free protein synthesis. Biotechnology and bioengineering 74, 309-316.
Kim, H.-C., Kim, T.-W., Kim, D.-M., (2011) Prolonged production of proteins in a cell-free protein synthesis system using polymeric carbohydrates as an energy source. Process Biochem 46, 1366-1369.
Kim, T.-W., Keum, J.-W., Oh, I.-S., Choi, C.-Y., Kim, H.-C., Kim, D.-M., (2007a) An economical and highly productive cell-free protein synthesis system utilizing fructose-1,6-bisphosphate as an energy source. J Biotechnol 130, 389-393.
Kim, T.-W., Oh, I.-S., Keum, J.-W., Kwon, Y.-C., Byun, J.-Y., Lee, K.-H., Choi, C.-Y., Kim, D.-M., (2007b) Prolonged cell-free protein synthesis using dual energy sources: Combined use of creatine phosphate and glucose for the efficient supply of ATP and retarded accumulation of phosphate. Biotechnol Bioeng 97, 1510-1515.
Ryabova, L. A., Vinokurov, L. M., Shekhovtsova, E. A., Alakhov, Y. B., Spirin, A. S., (1995) Acetyl phosphate as an energy source for bacterial cell-free translation systems. Analytical biochemistry 226, 184-186.
Schoborg, J. A., Hodgman, C. E., Anderson, M. J., Jewett, M. C., (2014) Substrate replenishment and byproduct removal improve yeast cell-free protein synthesis. Biotechnology journal 9, 630-640.
Shin, J., Noireaux, V., (2012) An E. coli cell-free expression toolbox:
application to synthetic gene circuits and artificial cells. ACS synthetic biology 1, 29-41.
Sun, Z. Z., Yeung, E., Hayes, C. A., Noireaux, V., Murray, R. M., (2014) Linear DNA for Rapid Prototyping of Synthetic Biological Circuits in an Escherichia coli Based TX-TL Cell-Free System. ACS Synth. Biol. 3, 387-397.
Swartz, J., (2006) Developing cell-free biology for industrial applications. Journal of industrial microbiology & biotechnology 33, 476-485.
Takahashi, M. K., Chappell, J., Hayes, C. A., Sun, Z. Z., Kim, J., Singhal, V., Spring, K. J., Al-Khabouri, S., Fall, C. P., Noireaux, V., Murray, R. M., Lucks, J. B., (2014) Rapidly Characterizing the Fast Dynamics of RNA Genetic Circuitry with Cell-Free Transcription-Translation (TX-TL) Systems. ACS synthetic biology.
Takai, K., Sawasaki, T., Endo, Y., (2010) Practical cell-free protein synthesis system using purified wheat embryos. Nat Protoc 5, 227-238.
Wang, Y., Zhang, Y. H. P., (2009) Cell-free protein synthesis energized by slowly-metabolized maltodextrin. BMC Biotechnol 9, 58.
Yin, G., Garces, E. D., Yang, J., Zhang, J., Tran, C., Steiner, A. R., Roos, C., Bajad, S., Hudak, S., Penta, K., Zawada, J., Pollitt, S., Murray, C. J., (2012) Aglycosylated antibodies and antibody fragments produced in a scalable in vitro transcription-translation system. mAbs 4, 217-225.
Zawada, J. F., Yin, G., Steiner, A. R., Yang, J., Naresh, A., Roy, S. M., Gold, D. S., Heinsohn, H. G., Murray, C. J., (2011) Microscale to manufacturing scale-up of cell-free cytokine production—a new approach for shortening protein production development timelines. Biotechnology and bioengineering 108, 1570-1578.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A reaction mixture for preparing a biological macromolecule in vitro, the reaction mixture comprising:
(a) a yeast cell-free extract;
(b) a phosphate-free energy source; and
(c) a phosphate source.

2. The reaction mixture of claim 1, wherein said phosphate-free energy source is selected from a group consisting of glucose, a glycolytic intermediate, a polymer comprising a glucose subunit, and any combination thereof.

3. The reaction mixture of claim 2, wherein the polymer comprising a glucose subunit is starch or dextran.

4. The reaction mixture of claim 2, wherein the glycolytic intermediate is selected from the group consisting of fructose 1,6-bisphosphate (FBP), phosphoenolpyruvate (PEP), glucose, 3-phosphglyceric acid (3-PGA), glucose 6-phosphate (G6P), and any combination thereof.

5. The reaction mixture of claim 1, wherein the phosphate source comprises exogenous phosphate.

6. The reaction mixture of claim 5, wherein exogenous phosphate is present in the reaction mixture at a concentration of from about 1 mM to about 30 mM.

7. The reaction mixture of claim 5, wherein exogenous phosphate is selected from a group consisting of potassium phosphate, magnesium phosphate and ammonium phosphate.

8. The reaction mixture of claim 1 further comprising cAMP.

9. The reaction mixture of claim 8, wherein cAMP is present in the reaction mix at a concentration of from about 0.05 mM to about 5 mM.

10. The reaction mixture of claim 1, wherein the yeast cell-free extract is a Saccharomyces cerevisiae cell-free extract.

11. The reaction mixture of claim 1, wherein the yeast cell-free extract is prepared from mid-exponential to late-exponential culture in the range from about 6 OD₆₀₀ to about 18 OD₆₀₀, or from a culture having a higher OD₆₀₀ where a fed-batch operation was performed.

12. The reaction mixture of claim 1, wherein the yeast cell-free extract is an S30 extract or an S60 extract.

13. The reaction mixture of claim 1 further comprising a reaction buffer.

14. The reaction mixture of claim 1 further comprising a translation template, a transcription template, or both a translation template and a transcription template.

15. The reaction mixture of claim 1 further comprising a polymerase capable of transcribing a transcription template to form a translation template.

16. The reaction mixture of claim 1, wherein the reaction mixture does not comprise an exogenous nucleoside triphosphate.

17. The reaction mixture of claim 1, wherein the biological macromolecule is an oligopeptide or a protein.

18. The reaction mixture of claim 16, wherein the biological macromolecule is an oligopeptide comprising a nonstandard amino acid subunit or a protein comprising a nonstandard amino acid subunit.

19. A method for synthesis of a biological macromolecule in vitro using yeast cell-free protein synthesis, the method comprising synthesizing the biological macromolecule from a translation template in a reaction mixture comprising: (i) a yeast cell-free extract; (ii) a phosphate-free energy source; and (iii) a phosphate source.

20. A kit for synthesizing a biological macromolecule in vitro using a cell-free protein synthesis system, the kit comprising as components: (i) a yeast cell-free extract; (ii) a phosphate-free energy source; and (iii) a phosphate source.
